(19) United States
(12) Patent Application Publication
(10) Pub. No.: US 2020/0360124 A1
(43) Pub. Date: Nov. 19, 2020

(54) TEETH WHITENING DEVICE

(71) Applicant: OSSTEMIMPLANT CO., LTD., Seoul (KR)

(72) Inventors: Jae Hee WOO, Suwon-si, Gyeonggi-do (KR); Min Soo GYEONG, Seoul (KR); Nam ii KIM, Seoul (KR)

(73) Assignee: OSSTEMIMPLANT CO., LTD., Seoul (KR)

(21) Appl. No.: 15/998,723

(22) PCT Filed: Feb. 13, 2017
(86) PCT No.: PCT/KR2017/001548

§ 371 Date: Aug. 16, 2018

(30) Foreign Application Priority Data
Feb. 16, 2016 (KR) 10-2016-0017634

(51) Int. Cl. A61C 19/06; A61N 5/06

(52) U.S. Cl. CPC A61C 19/06; A61N 5/0601

(57) ABSTRACT

Provided is a teeth whitening device. The device includes: a power supply unit supplying power; a light irradiation unit irradiating teeth whitening light by using the power; and an oral wearing unit detachably coupled to the light irradiation unit by a magnetic force and inserted into an oral cavity of a user in use.

### TECHNICAL FIELD

The present invention relates to a teeth whitening device, and more particularly, to a teeth whitening device that irradiates light to a tooth coated with a whitening agent so as to exert a teeth whitening function through a photocatalyst.

### BACKGROUND ART

Recently, many people have been interested in well-being. Industrialization has made people enjoy material abundance, and now they have much interest in how to live a better life than what to eat. As the level of living improves, people try to change their external appearance beautifully as a way to make their lives more beautiful. One example among the ways is teeth whitening that makes teeth of a user be whitened, which can give a clean image of the user to people which the user meets.

At the beginning of teeth whitening, a whitening effect is obtained by tooth brushing when brushing with toothpaste containing a small amount of teeth whitening agent as the simplest way.

However, since the amount of the whitening agent contained in the toothpaste is too small, a coloring component deposited on a tooth surface cannot be effectively removed.

For more effective whitening treatment, there is also a method of receiving professional treatment using a whitening pad or laser in the dentist. Such a method is excellent in the whitening effect, but it is troublesome to receive outpatient treatment periodically for a long period of time and expensive treatment cost is required.

As a result, a whitening patch has been proposed as an eclectic alternative which is simple and easy to handle and is high effectively with less cost.

The whitening patch is a product which is coated with an ointment agent containing a whitening agent component based on a low concentration of carboxide and peroxide on the surface thereof and has a sufficient whitening component and can be used for a long time to exert the whitening effect and it is possible to use the whitening patch simply because it is ready for treatment only by attaching to the front and back of the teeth.

However, the whitening patch is detached from the original attachment position due to movement of the muscles around the mouth when used for a long time because an adhesive portion adhered to the tooth is water-soluble. The whitening patch has a problem that the ointment portion irritates or damages the gum, tongue, or oral mucosa.

Recently, teeth whitening devices have been developed to overcome the problems. The teeth whitening device is constituted by a mouth opening tool inserted into an oral cavity and irradiating the teeth with light and a main body having a switch connected to the mouth opening tool through a cable to supply power and control ON/OFF.

However, the mouth opening tool of the teeth whitening device in the related art is worn on the oral cavity after the whitening agent is applied to the tooth due to a structural characteristic thereof. In this case, the oral cavity/lips other than the teeth are smeared with the teeth whitening agent applied to the teeth and the whitening agent is thus removed before the whitening.

In addition, the power supply means of the teeth whitening device in the related art is implemented in a shape difficult to wear on the user's body, and is stored in a pocket of a user in use. In this case, due to a wired cable connecting the power supply means and the mouth opening tool, the movement of the user is restricted, which causes inconvenience in use.

In addition, since the teeth whitening device in the related art has a structure in which heat generated by light irradiation is difficult to be emitted, there is a disadvantage that the user may feel uncomfortable when using the teeth whitening device for a long time.

Further, there has been a problem that air discharged while the user who wears the mouth opening tool breathes, in particular, discharging the breath by exhalation which is sprayed around the mouth of the user, thereby increasing the user's discomfort.

### DISCLOSURE

### Technical Problem

The present invention is contrived to solve the above-mentioned problems, and an object of the present invention is to provide a teeth whitening device formed so that a light irradiation unit irradiating teeth whitening light and an oral wearing unit inserted into an oral cavity of the user in use are formed to be attached/detached to/from each other by a magnetic force.

Further, another object of the present invention is to provide a teeth whitening device which includes a strap having a predetermined length so as to be hung in a part of the body of the user when a power supply unit is used.

In addition, yet another object of the preset invention is to provide a teeth whitening device in which a through-hole is formed in the oral wearing unit, at least one vent groove is formed on a coupling surface of the light irradiation unit and/or the oral wearing unit, and the through-hole and the vent groove constitute a passage of an air flow.

### Technical Solution

In order to achieve the objects, a teeth whitening device according to an embodiment of the present invention includes: a power supply unit supplying power; a light irradiation unit irradiating teeth whitening light by using the power; and an oral wearing unit detachably coupled to the light irradiation unit by a magnetic force and inserted into an oral cavity of a user in use.

In addition, at least one magnetic force member providing the magnetic force may be formed on a coupling surface of each of the light irradiation unit and the oral wearing unit.

Further, the oral wearing unit may comprise a close contact member closely contacting gums of the user, a coupling member detachably coupled to a cover of the light irradiation unit, and a holding member formed between the close contact member and the coupling member and holding lips of the user, and at least one first magnetic force member providing the magnetic force may be formed in the coupling member.

In addition, the first magnetic force member may be formed to protrude on an exterior of the coupling member.

Further, at least one insertion groove may be formed and a second magnetic force member which is able to be coupled with the first magnetic force member may be formed inside the insertion groove.

In addition, the oral wearing unit may further include two bite members formed to protrude on the exterior of the close contact member so as to bite the teeth whitening device with teeth when the teeth whitening device is inserted into the oral cavity of the user.

Further, a through-hole penetrating a front surface and a rear surface may be formed at a central portion of the oral wearing unit.

In addition, at least one vent groove may be formed on the coupling surface of the light irradiation unit and/or the oral wearing unit.

Further, when the light irradiation unit and the oral wearing unit are coupled to each other, the through-hole and the vent groove may form a passage of an air flow depending on inhalation or exhalation of the user.

In addition, the vent groove may be formed at a position so as to prevent air discharged by the exhalation from moving to a body of the user.

Further, a first vent groove may be formed at a left side and a second vent groove may be formed at a right side of the coupling surface of the light irradiation unit, a third vent groove may be formed at the left side and a fourth vent groove may be formed at the right side of the coupling surface of the oral wearing unit, and when the light irradiation unit and the oral wearing unit are coupled to each other, the first and third vent grooves may form the passage and the second and fourth vent grooves may form the passage.

In addition, the light irradiation unit may include a cover constituted by a front cover and a rear cover, a light source substrate provided with a light source and mounted between the rear cover and the front cover, and a protection film covering one side of the light source substrate so as to protect the light source of the light source substrate.

Further, the power supply unit may include a power supply unit body having a power supply module for supplying power, and a strap having a predetermined length so as to be hung on a part of the body of the user.

In addition, a magnetic force member may be provided on at least one of both ends of the strap, and one of both ends of the strap may be detachably coupled to the power supply unit body by the magnetic force.

Further, the light irradiation unit and the power supply unit may be connected to each other by wire or wirelessly, and the light irradiation unit may receive a turn-on control signal and the power of the light source from the power supply unit through the connection.

### Advantageous Effects

According to various embodiments of the present invention described above, the light irradiation unit and the oral wearing unit are formed so as to be detachable from each other by the magnetic force, so that the oral wearing unit is first inserted into the oral cavity to fix the mouth in an open state and then the teeth whitening agent is applied and the light irradiation unit can be then bonded to the oral wearing unit. As a result, the teeth whitening agent can be applied to teeth more smoothly, which is advantageous in solving the problems in the related art. In addition, since the user can move the light irradiation unit to the vicinity of the fixed oral wearing unit and place the light irradiation unit within the influence of the magnetic force, the oral wear unit and the light irradiation unit can be combined with each other.

Further, according to various embodiments of the present invention described above, since the power supply unit is formed to be partly wearable to the user's body, there is an advantage in that it is easy to carry and the activity can be freely performed even while whitening teeth.

In addition, according to various embodiments of the present invention described above, a structure is provided, in which heat generated from a light source can be effectively discharged through a vent hole formed at the mouth opening tool and oxygen can be smoothly supplied without discomfort to a user, and as a result, the teeth whitening device can be used for a long time.

### DESCRIPTION OF DRAWINGS

FIG. 1 is a block diagram illustrating a teeth whitening device according to an embodiment of the present invention.

FIG. 2 is a perspective view illustrating a teeth whitening device according to an embodiment of the present invention.

FIG. 3 is a perspective view illustrating a use state of a power supply unit according to an embodiment of the present invention.

FIG. 4 is a perspective view illustrating a mouth opening tool according to an embodiment of the present invention.

FIG. 5 is an exploded perspective view illustrating the mouth opening tool according to an embodiment of the present invention.

FIG. 6 is a perspective view illustrating a coupling surface of a light irradiation unit and an oral wearing unit according to an embodiment of the present invention.

FIG. 7 is a perspective view illustrating an attachment/detachment state of the light irradiation unit and the oral wearing unit according to an embodiment of the present invention.

FIG. 8 is a cross-sectional view illustrating an air passage of the mouth opening tool according to an embodiment of the present invention.

### MODE FOR INVENTION

The matters relating to the functional effects of the teeth whitening device according to the present invention including the technical structure of the above object will be clearly understood by the following detailed description referring to drawings in which preferred embodiments of the present invention are illustrated.

Advantages and features of the present invention, and techniques for accomplishing the same will be more clearly understood from exemplary embodiments described in detail below with reference to the accompanying drawings. However, the present disclosure is not limited to an embodiment disclosed below but may be implemented in various different forms. The present embodiments are provided so that the disclosure of the present invention may not only be complete, but also may fully provide the scope of the present invention to those skilled in the art. Throughout the whole specification, the same reference numerals denote the same elements.

For simplicity and clarity of illustration, the drawings illustrate a general configuration scheme and a detailed description of known features and techniques may be omitted so as to avoid unnecessarily obscuring the discussion of the described embodiments of the present invention. Additionally, components of the drawings are not necessarily drawn according to a scale. For example, to facilitate understanding of embodiments of the present invention, some of the elements of the figures may be exaggerated relative to other elements. Like reference numerals in different drawings denote like elements, and similar reference numerals can indicate similar elements, although not necessarily.

Hereinafter, preferred embodiments of the present invention will be described in detail with reference to the accompanying drawings, in which like reference numerals refer to like or corresponding elements regardless of reference numerals and a duplicated description thereof will be omitted.

In describing the present invention, a detailed description of related known technologies will be omitted if it is determined that they make the gist of the present invention unclear.

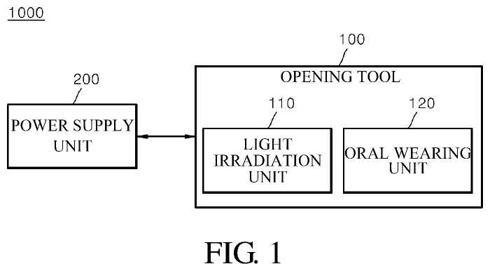

FIG. 1 is a block diagram illustrating a teeth whitening device according to an embodiment of the present invention. Referring to FIG. 1, a teeth whitening device 1000 may include a mouth opening tool 100 and a power supply unit 200.

The mouth opening tool 100 is configured to be worn on an oral cavity of a user in use. Specifically, the mouth opening tool 100 may open and fix a mouth of the user, may be inserted into the oral cavity of the user, and may supply light to teeth of the user.

The mouth opening tool 100 may include a light irradiation unit 110 for irradiating teeth whitening light by using power of the power supply unit 200 and an oral wearing unit 120 inserted into the oral cavity of the user. Here, the light irradiation unit 110 and the oral wearing unit 120 may be detachably coupled to each other by a magnetic force.

The power supply unit 200 may supply the power to respective components constituting the mouth opening tool 100. Specifically, the power supply unit 200 may supply the power to the light irradiation unit 110 irradiating the teeth whitening light.

Further, the power supply unit 200 may generate a light source turn-on control signal and may transmit the generated control signal to the mouth opening tool 100. The light irradiation unit 110 may not irradiate the light or irradiate the light according to the light source turn-on control signal.

Here, the power supply unit 200 is implemented as a rechargeable battery and may be thus charged by receiving charging current when a remaining power amount is insufficient.

The power supply unit 200 may be implemented as an internal power source provided inside a housing of the mouth opening tool. In this case, the internal power source may be detachable within the housing or may not be detachable.

Alternatively, the power supply unit 200 may be implemented as an external power source such as an external battery disposed outside the housing of the mouth opening tool.

If the power supply unit 200 is implemented as the external power source, the mouth opening tool 100 and the power supply unit 200 may be connected by wire or wireless.

Further, when the power supply unit 200 is implemented as the external power source, the power supply unit 200 may be implemented in a form in which the power supply unit 200 may be worn on the body of the user.

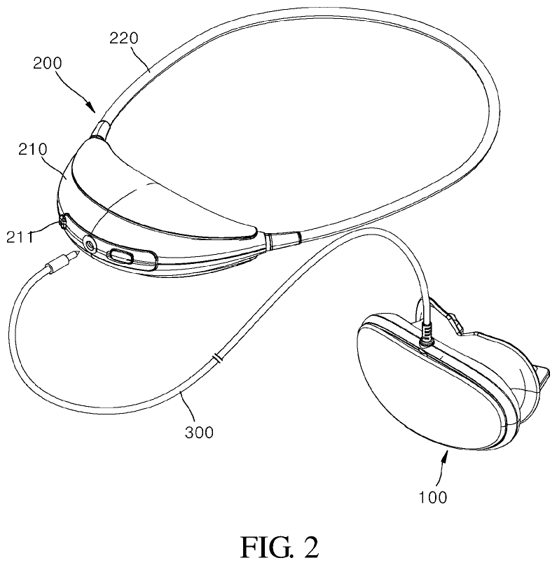

Hereinafter, with reference to FIG. 2, the teeth whitening device 1000 according to an embodiment of the present invention will be described in more detail.

FIG. 2 is a perspective view illustrating a teeth whitening device according to an embodiment of the present invention. Referring to FIG. 2, the mouth opening tool 100 and the power supply unit 200 may be connected to each other by wire through a cable 300. At least one of the mouth opening tool 100 and the power supply unit 200 may include a connector to which the cable 300 is connected. In this case, the power supply unit 200 may supply the turn-on control signal and the power to the mouth opening tool 100 through the cable 300.

The power supply unit 200 may include a power supply unit body 210 having a power supply module for supplying the power, for example, a battery, a battery charging circuit, etc., and a strap 220 having a predetermined length so as to be hung on a part of the body of the user.

Here, a button 211 for controlling the turn-on control signal may be provided on one side of the power supply unit body 210. In this case, depending on a user input through the button 211, the light irradiation unit 110 of the mouth opening tool 100 may irradiate the light or not irradiate the light.

The strap 220 may have the predetermined length so as to be hung on a part of the body of the user. As an example, as illustrated in FIG. 2, the strap 220 may be implemented in a length in which the strap 200 may be hung on a neck. However, this is just an embodiment of the present invention and may be implemented in various forms such as a bracelet type, an anklet type, and the like by varying the length thereof.

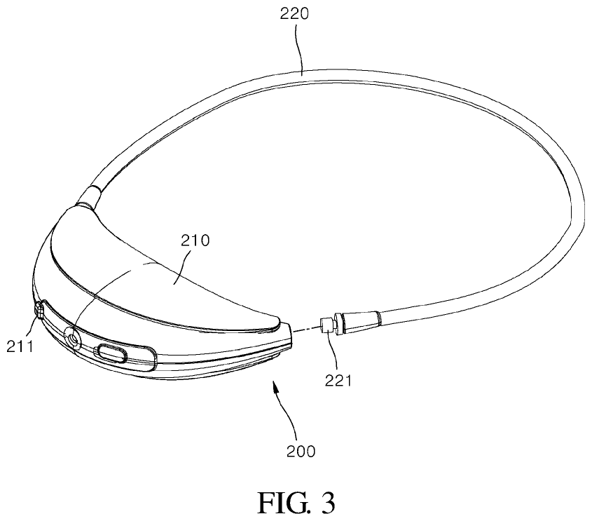

Meanwhile, at least one of both ends of the strap 220 may be detachably coupled to the power supply unit body 210 for the convenience of wearing on the body of the user. This will be described in more detail with reference to FIG. 3.

FIG. 3 is a perspective view illustrating .a use state of a power supply unit according to an embodiment of the present invention. Referring to FIG. 3, at least one of both ends of the strap 220 may be provided with a magnetic force member 221. In addition, a magnetic force member (not illustrated) may be provided at a coupling portion of the strap 220 of the power supply unit body 210.

In this case, one of both ends of the strap 220 may be detachably coupled to the power supply unit body 210 by the magnetic force.

Accordingly, when using the teeth whitening device 1000, the user may easily wear the strap 220 on a part of the body thereof while separating one end of the strap 220 from the power supply unit body 210. Further, when wearing the strap 220 is completed, the user attaches one end of the strap 220 to the power supply unit body 210 to hang the strap 220 on a part of the body thereof, and as a result, the user may perform a free activity even while teeth whitening.

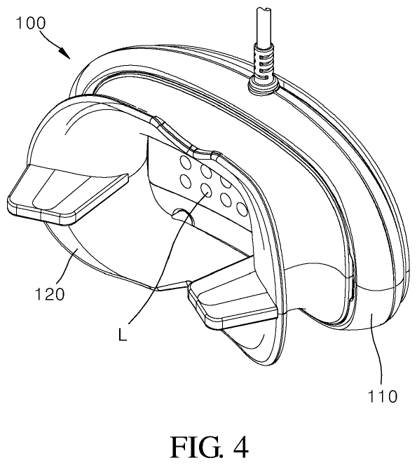

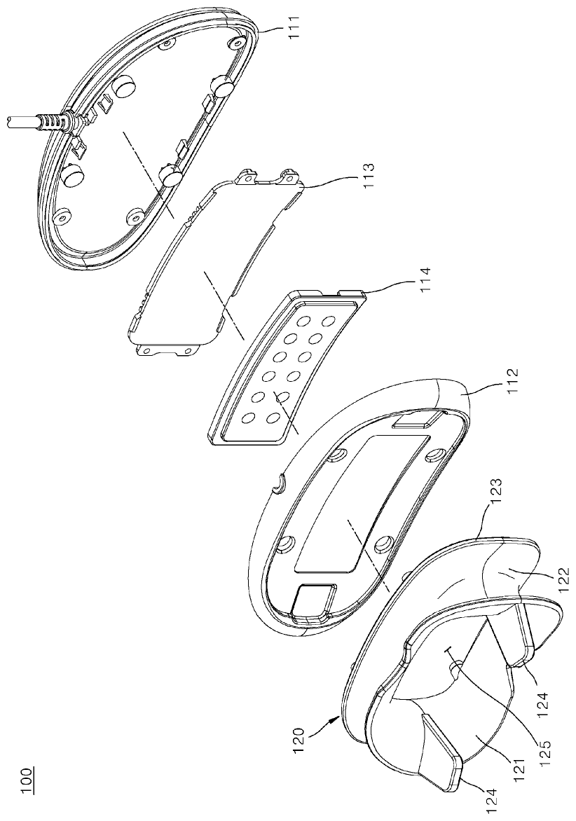

Hereinafter, with reference to FIGS. 4 and 5, the mouth opening tool according to an embodiment of the present invention will be described in more detail.

FIG. 4 is a perspective view illustrating a mouth opening tool according to an embodiment of the present invention. FIG. 5 is an exploded perspective view illustrating the mouth opening tool according to an embodiment of the present invention. Referring to FIGS. 4 and 5, the mouth opening tool 100 may include the light irradiation unit 110 for irradiating the teeth whitening light by using the power of the power supply unit 200 and the oral wearing unit 120 inserted into the oral cavity of the user.

The light irradiation unit 110 includes a light source L and may irradiate the teeth with the whitening light through the through-hole of the oral wearing unit 120. Here, the light irradiation unit 110 may be implemented in various shapes including an ellipse, a circle, a square, and the like.

Here, the light irradiation unit 110 includes a rear cover 111, an annular front cover 112 mounted on a front portion of the rear cover 111, and the light source L such as an LED on one side and may include a light source substrate 113 mounted between the rear cover 111 and the front cover 112, and a protection film 114 covering one side of the light source substrate 113 so as to protect the light source L of the light source substrate 111 from moisture and the like.

The front cover 112 is formed in an annular shape and is mounted on the front portion of the rear cover 111 so that the light source substrate 113 may be mounted between the rear cover 111 and the front cover 112, and the teeth whitening light irradiated from the light source L may be irradiated to the teeth of the user through an opening of the front cover 112.

Here, the rear cover 111 and the front cover 112 may be made of a synthetic material, plastic (e.g. ABS, polycarbonate), ceramic, wood, metal (e.g. polished aluminum), metal alloy (e.g., steel, stainless steel, titanium or magnesium based alloys), or other suitable materials.

The light source substrate 113 is a substrate having an LED light source L for irradiating the teeth whitening light to the teeth of the user and the LED light source L mounted on the light source substrate 113 may adopt LEDs having various colors and illuminance.

The protection film 114 may be provided on the surface of the light source substrate 113 to protect the light source L from the moisture so that the LED light source L may be protected from a moist environment in the oral cavity. The protection film 114 may be selectively replaced by the user.

Meanwhile, the oral wearing unit 120 is preferably made of a soft material such as silicone which is harmless to the human body since the oral wearing unit 120 is in direct contact with the teeth and the gums and may be curved in an arc shape so as to surround an entire outer surface of the teeth. Here, a through-hole 125 may be formed at a central portion of the oral wearing unit 120 and the through-hole 125 may be formed through a front surface and a rear surface of the oral wearing unit 120.

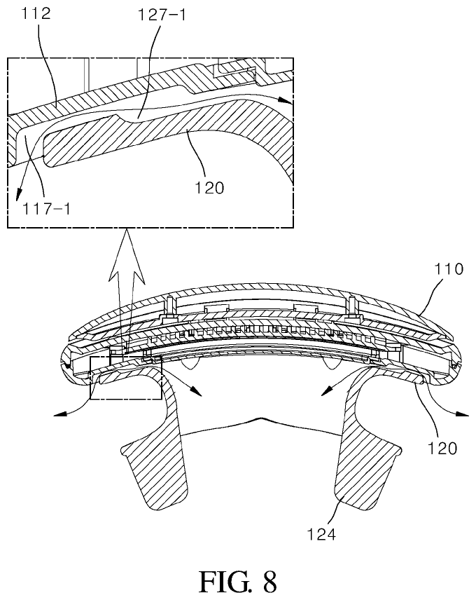

The through-hole 125 may form a passage through which the light source L of the light irradiation unit 110 is irradiated to the teeth of the user. In addition, the through-hole 125 may form a passage for emitting heat generated according to the irradiation of the light and a passage of an air flow due to inhalation or exhalation of the user, or the like, in combination with a vent groove of the light irradiation unit 110 and/or the oral wearing unit 120. This will be described below with reference to FIG. 8.

Meanwhile, the oral wearing unit 120 may include a close contact member 121 which is brought into close contact with the gums of the user, a coupling member 123 which is detachably coupled to a cover of the light irradiation unit 110, and a holding member 122 formed between the close contact member 121 and the coupling member 123 and held with lips of the user. In addition, the oral wearing unit 120 may further include two bite members 124 protruding on an exterior of the close contact member so as to be held in the teeth when the oral cavity of the user is inserted.

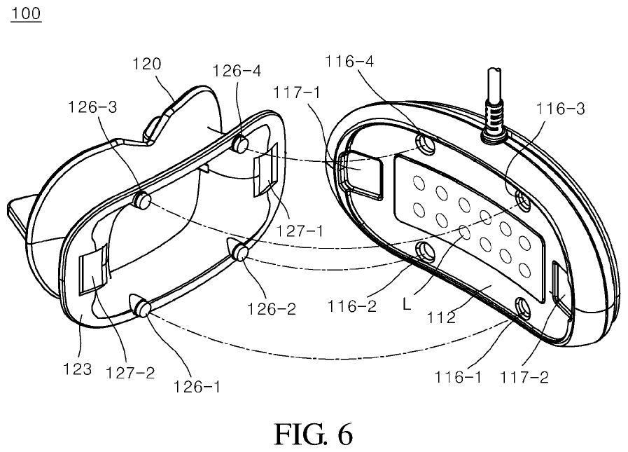

Meanwhile, at least one magnetic force member providing the magnetic force may be formed on a coupling surface coupled with the oral wearing unit 120 in use among a plurality of surfaces of the light irradiation unit. Further, at least one magnetic force member providing the magnetic force may be formed on a coupling surface coupled with the light irradiation unit 110 in use among a plurality of surfaces of the oral wearing unit 120. As a result, the light irradiation unit 110 and the oral wearing unit 120 may be detachably coupled to each other by the magnetic force. This will be described in detail with reference to FIG. 6.

FIG. 6 is a perspective view illustrating a coupling surface of a light irradiation unit and an oral wearing unit according to an embodiment of the present invention. Referring to FIG. 6, at least one of first magnetic force members 126-1, 126-2, 126-3, and 126-4 for providing the magnetic force may be formed on the coupling surface of the coupling member 123. The first magnetic force members 126-1, 126-2, 126-3, and 126-4 may be formed to protrude on the exterior of the coupling member 123.

Further, one or more insertion grooves 116-1, 116-2, 116-3, and 116-4 are formed on the coupling surface of the front cover 112 and second magnetic force members which may be coupled with the first magnetic force members 126-1, 126-2, 126-3, and 126-4 may be formed inside insertion grooves 116-1, 116-2, 116-3, and 116-4.

Thus, a 1-1^{st} magnetic force member 126-1 is magnetically coupled with a 2-1^{st} magnetic force member and inserted into a first insertion groove 116-1, a 1-2^{nd} magnetic force member 126-2 is magnetically coupled with a 2-2^{nd} magnetic force member and inserted into a second insertion groove 116-2, a 1-3^{rd} magnetic force member 126-3 is magnetically coupled with a 2-3^{rd} magnetic force member and inserted into a third insertion groove 116-3, and a 1-4^{th} magnetic force member 126-4 is magnetically coupled with a 2-4^{th} magnetic force member and inserted into a fourth insertion groove 116-4. That is, the light irradiation unit 110 and the oral wearing unit 120 may be detachably coupled to each other by the magnetic force.

Meanwhile, in FIG. 6, the number of magnetic force members and the shape of the magnetic force member are just for convenience of description and are not limited thereto. Therefore, the number of magnetic force members, the protrusion of the magnetic force member, and the insertion or not of the magnetic force members may be designed and changed in various forms according to the implementation method of a structure for magnetic coupling.

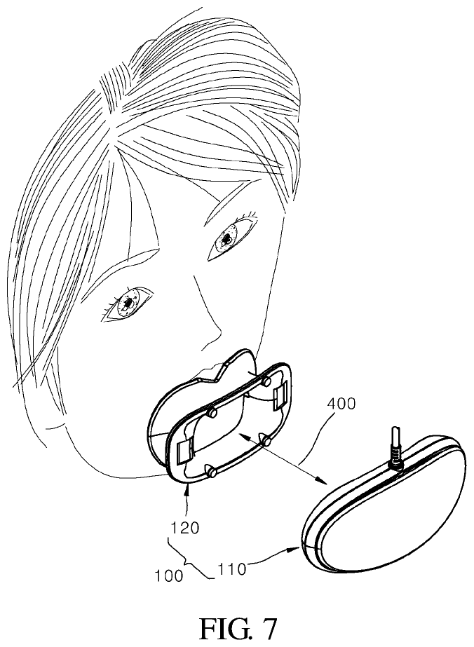

Meanwhile, according to a detachable structure of the light irradiation unit 110 and the oral wearing unit 120 by the magnetic force, user convenience of the user may be enhanced. This will be described in more detail with reference to FIG. 7.

FIG. 7 is a perspective view illustrating an attachment/detachment state of the light irradiation unit and the oral wearing unit according to an embodiment of the present invention. Referring to FIG. 7, first, the user may insert the oral wearing unit 120 into the oral cavity and holds the lips on a holding member 122 to fix the mouth while opening the mouth. In this case, since the mouth is fixed in an open state, the user may more smoothly apply the teeth whitening agent to the teeth.

When the application of the teeth whitening agent is completed, the user may couple the light irradiation unit 110 to the oral wearing unit 120. In this case, the user moves the light irradiation unit 110 near the fixed oral wearing unit 120 and place the light irradiation unit 110 within the influence of a magnetic force 400 to couple the oral wearing unit 120 and the light irradiation unit 110, and as a result, the oral wearing unit 120 and the light irradiation unit 110 may be more easily coupled.

Meanwhile referring to FIG. 6, a through-hole 125 may be formed at a central portion of the oral wearing unit 120. In addition, one or more vent grooves 117-1 and 117-2 may be formed on the coupling surface of the front cover 112. In addition, one or more vent grooves 127-1 and 127-2 may be formed on the coupling surface of the coupling member 123.

The vent grooves 117-1, 117-2, 127-1, and 127-2 may be formed at positions where the air discharged from the inside of the body does not face the body of the user when the user exhales. For example, the vent grooves 117-1, 117-2, 127-1, and 127-2 may be formed on the side of the coupling surface.

More specifically, a first vent groove 117-1 may be formed on a left side of the coupling surface of the front cover 112, a second vent groove 117-2 may be formed on a right side, a third vent groove 127-1 may be formed on the left side of the coupling surface of the coupling member 123, and a fourth vent groove 127-2 may be formed on the right side.

When the light irradiation unit 110 and the oral wearing unit 120 are coupled to each other by the magnetic force, the first vent groove 117-1 and the third vent groove 127-1 may form a first passage for the flow of the air and the second vent groove 117-2 and the fourth vent groove 127-2 may form a second passageway for the flow of the air.

Meanwhile, in FIG. 6, the number of vent grooves is just for convenience of description and is not limited thereto. Thus, the number of vent grooves may be varied in design in various forms depending on an implementation scheme of a structure for ventilation.

In FIG. 6, the position of the vent groove is just a preferred embodiment of the present invention for preventing the air discharged from moving toward the body of the user when the user exhales, but is not limited thereto. Thus, the position of the vent groove may be varied in design in various forms depending on the implementation scheme of a structure for ventilation.

Meanwhile, when the irradiation unit 110 and the oral wearing unit 120 are coupled to each other by the magnetic force, the through-hole 125 and the vent groove may form a passage for discharge of heat generated as irradiation of the light and a passage of the air flow depending on the inhalation or exhalation of the user. This will be described in detail with reference to FIG. 8.

FIG. 8 is a perspective view illustrating an air passage of the mouth opening tool according to an embodiment of the present invention. Referring to FIG. 8, when the light irradiation unit 110 and the oral wearing unit 120 are coupled to each other by the magnetic force, a first passage formed by coupling the first vent groove 117-1 and the third vent groove 127-1, a second passage formed by coupling the second vent groove 117-2 and the fourth vent groove 127-2, and a third passage formed by the through-hole 125 may be connected to each other. That is, the first passage, the second passage, and the third passage may be connected to each other to form the passage for the discharge of the heat generated according to the irradiation of the light and the passage for the airflow depending on the inhalation or exhalation of the user.

Accordingly, the air G depending on the exhalation of the user who wears the mouth opening tool 100 may be discharged to the outside through the passage and the air G depending on the inhalation of the user may flow into lungs of the user through the passage.

Meanwhile, an air passage may release the heat generated in the light source L through the flow of air (G) caused by the inhalation or exhalation of the user who breathes while whitening the teeth by wearing the mouth opening tool 100.

In particular, when the user exhales, the air U discharged from the inside of the body is discharged through the air passage and in this case, since the heat generated by the LED light source L is discharged to the outside together with the air G, the light source L may be cooled.

In addition, when the user inhales, external air (G) containing oxygen flows into the inside of the body through the air passage, through which oxygen is supplied to the tooth coated with the whitening agent, so that the whitening function of the photocatalyst may be promoted and the LED light source L may be cooled as the heat generated from the LED light source L flows into the inside of body together with the air G.

According to the present invention, a structure is provided, in which the heat generated from the light source may be effectively discharged through the vent groove formed at the mouth opening tool and the oxygen may be smoothly supplied without discomfort to the user, and as a result, the teeth whitening device may be used for a long time.

Meanwhile, a teeth whitening process using the teeth whitening device according to an embodiment of the present invention described above will be described below.

<Teeth Whitening Process>

First step: First, the user may insert the oral wearing unit 120 into the oral cavity, bite two bite members 124 with teeth, and hold the lips on the holding member 122. In this case, the mouth of the user may be fixed in the open state.

Second step: The user may directly apply the whitening agent to the teeth inside the opened mouth. Here, a teeth whitening composition of the whitening agent may contain a composition containing an active ingredient capable of decoloring teeth coloring chromophores or reducing coloring of a dyed chromophore, or a composition capable of dissolving a dyed chromophore to be more susceptible to mechanical or chemical removal. As an example, the active ingredient may include hydrogen peroxide (or any hydrogen peroxide precursor), carbamide peroxide, sodium percarbonate, calcium peroxide, sodium perborate, potassium peracetate, peracetic acid (or other peracids), and chlorine dioxide.

Third step: The user may move the light irradiation unit 110 to the vicinity of the oral wearing unit 120 and place the light irradiation unit 110 within the influence of the magnetic force 400, thereby coupling the oral wearing unit 120 and the light irradiation unit 110.

Fourth step: In order to initiate teeth whitening, the user may press the button 211 of the power supply unit and in this case, the light irradiation unit 110 may irradiate the light toward the teeth. Accordingly, a photoactive agent contained in the whitening agent is activated by light energy of the irradiation light to promote a whitening reaction of a whitening component, thereby shortening a whitening treatment time and increasing a whitening effect. Here, the irradiation light may be LED light of 400 nm to 505 nm which is the most suitable wavelength range for teeth whitening and in this case, the functions of sterilization and teeth whitening may be performed in parallel. However, the wavelength range of the irradiation light is not limited to the above example.

Fifth step: After irradiating light for a predetermined time, the user may press the button 211 of the power supply unit to terminate the teeth whitening. In this case, the light irradiation unit 110 may stop the light irradiation. Here, the predetermined period may be approximately 5 minutes to 10 minutes, but is not limited thereto.

Meanwhile, in the specification and claims, terms such as “first”, “second”, “third”, and “fourth” are used to distinguish similar components from each other, even though it is not particularly so, the terms are used to describe a specific order or occurrence sequence. It will be appreciated that the terminology used is such that the embodiments of the invention described herein are compatible under the right circumstances, for example, so as to be able to operate in a sequence other than those shown or described herein. Likewise, where the method is described as including a series of steps, the order of such steps presented herein is not necessarily the order in which such steps may be performed, any of the described steps may be omitted and/or any other steps not described will be additive to the method.

Further, it is also to be understood that the terms “left”, “right”, “front”, “rear”, “upper”, “bottom”, “above”, “below, etc., are used for description, but are not particularly intended to describe an immutable relative position. It will be appreciated that the terminology used is such that the embodiments of the invention described herein are compatible under the right circumstances, for example, so as to be able to operate in a direction other than those shown or described herein. The term “connected”, as used herein, is defined as being directly or indirectly connected in an electrically or non-electrical manner. Objects described herein as “adjacent” to one another may be in physical contact with one another, in close proximity to one another, or in the same general range or region as are appropriate for the context in which the phrase is used. The presence of the phrase “in an embodiment” herein means the same embodiment, although not necessarily.

It is also to be understood that designation of ‘connected’, ‘connecting’, ‘fastened’, ‘fastening’, ‘coupled’, ‘coupling’, etc., and various modifications of the expressions in the specification and claims is used as a meaning including ‘directly connected with another component’ or ‘indirectly connected through another component’.

Further, suffixes “module” and “unit” for components used in the present specification are given or mixed in consideration of easy preparation of the specification only and do not have their own distinguished meanings or roles.

It is also to be understood that the terminology used herein is for the purpose of describing embodiments only and is not intended to limit the present invention. In this specification, singular forms include even plural forms unless the context clearly dictates otherwise. It is to be understood that the terms ‘comprise’ and/or ‘comprising’ used in the specification are intended to be inclusive in a manner that does not exclude the presence of stated components, steps, operations, and/or elements does not exclude the addition.

The present invention has been described above with reference to preferred embodiments thereof. All embodiments and conditional examples disclosed herein are intended to assist those skilled in the art to understand the principle and the concept of the present invention and it will be appreciated by those skilled in the art that the present invention may be implemented in a modified form without departing from the essential characteristics of the present invention.

Therefore, the disclosed embodiments should be considered in an illustrative viewpoint rather than a restrictive viewpoint. The scope of the present invention is defined by the appended claims rather than by the foregoing description, and all differences within the scope of equivalents thereof should be construed as being included in the present invention.

1. A teeth whitening device comprising:
a power supply unit supplying power;
a light irradiation unit irradiating teeth whitening light by using the power; and
an oral wearing unit detachably coupled to the light irradiation unit by a magnetic force and inserted into an oral cavity of a user in use.

2. The teeth whitening device of claim 1, wherein at least one magnetic force member providing the magnetic force is formed on a coupling surface of each of the light irradiation unit and the oral wearing unit.

3. The teeth whitening device of claim 1, wherein the oral wearing unit comprises a close contact member closely contacting gums of the user, a coupling member detachably coupled to a cover of the light irradiation unit, and a holding member formed between the close contact member and the coupling member and holding lips of the user, and
at least one first magnetic force member providing the magnetic force is formed in the coupling member.

4. The teeth whitening device of claim 3, wherein the first magnetic force member is formed to protrude on an exterior of the coupling member.

5. The teeth whitening device of claim 4, wherein on the cover of the light irradiation unit, at least one insertion groove is formed and a second magnetic force member which is able to be coupled with the first magnetic force member is formed inside the insertion groove.

6. The teeth whitening device of claim 3, wherein the oral wearing unit further includes two bite members formed to protrude on the exterior of the close contact member so as to bite the teeth whitening device with teeth when the teeth whitening device is inserted into the oral cavity of the user.

7. The teeth whitening device of claim 1, wherein a through-hole penetrating a front surface and a rear surface is formed at a central portion of the oral wearing unit.

8. The teeth whitening device of claim 7, wherein at least one vent groove is formed on the coupling surface of the light irradiation unit and/or the oral wearing unit.

9. The teeth whitening device of claim 8, wherein when the light irradiation unit and the oral wearing unit are coupled to each other, the through-hole and the vent groove form a passage of an air flow depending on inhalation or exhalation of the user.

10. The teeth whitening device of claim 9, wherein the vent groove is formed at a position so as to prevent air discharged by the exhalation from moving to a body of the user.

11. The teeth whitening device of claim 8, wherein a first vent groove is formed at a left side and a second vent groove is formed at a right side of the coupling surface of the light irradiation unit,
a third vent groove is formed at the left side and a fourth vent groove is formed at the right side of the coupling surface of the oral wearing unit, and
when the light irradiation unit and the oral wearing unit are coupled to each other, the first and third vent grooves form the passage and the second and fourth vent grooves form the passage.

12. The teeth whitening device of claim 1, wherein the light irradiation unit includes,
a cover constituted by a front cover and a rear cover,
a light source substrate provided with a light source and mounted between the rear cover and the front cover, and
a protection film covering one side of the light source substrate so as to protect the light source of the light source substrate.

13. The teeth whitening device of claim 1, wherein the power supply unit includes
a power supply unit body having a power supply module for supplying power, and
a strap having a predetermined length so as to be hung on a part of the body of the user.

14. The teeth whitening device of claim 13, wherein a magnetic force member is provided on at least one of both ends of the strap, and
one of both ends of the strap is detachably coupled to the power supply unit body by the magnetic force.

15. The teeth whitening device of claim 1, wherein the light irradiation unit and the power supply unit are connected to each other by wire or wirelessly, and
the light irradiation unit receives a turn-on control signal and the power of the light source from the power supply unit through the connection.
